(19) United States
(12) Patent Application Publication
(10) Pub. No.: US 2011/0263994 A1
(43) Pub. Date: Oct. 27, 2011

(54) Method and Apparatus for Acquiring and Analyzing Data Relating to a Physiological Condition of a Subject

(76) Inventors: James Alexander Burns, British Columbia (CA); Graeme Jahns, Bristish Columbia (CA); David Lancaster, Bristish Columbia (CA); David MacQuarrie, Bristish Columbia (CA); Mark Miller, Bristish Columbia (CA); Kimora Rotherie, Bristish Columbia (CA); Max Windels, Bristish Columbia (CA)

(21) Appl. No.: 12/747,891

(22) PCT Filed: Dec. 11, 2008
(86) PCT No.: PCT/CA08/02201

§ 371 Date: Jan. 14, 2011

Related U.S. Application Data
(60) Provisional application No. 61/107,646, filed on Oct. 22, 2008.
(60) Provisional application No. 61/107,643, filed on Oct. 22, 2008.
(60) Provisional application No. 61/013,641, filed on Dec. 14, 2007.
(60) Provisional application No. 61/006,016, filed on Dec. 14, 2007.
(60) Provisional application No. 61/013,636, filed on Dec. 13, 2007.
(60) Provisional application No. 61/013,307, filed on Dec. 13, 2007.

(51) Int. Cl. A61B 5/02

(52) U.S. Cl. 600/509

(57) ABSTRACT

A method for locating and marking points on a waveform includes providing data corresponding to electrocardiogram and ballistocardiogram waveforms correlated in time, searching the data to locate points corresponding to cardiac events, a location of each of the points corresponding to cardiac events being defined by a rule set, identifying and storing the points corresponding to cardiac events and outputting a visual representation including the points corresponding to cardiac events marked on the electrocardiogram and ballistocardiogram waveforms.

### TECHNICAL FIELD

The present invention relates to a method and apparatus for acquiring and analyzing data relating to a physiological condition of a subject, in particular, a method and apparatus for acquiring and analyzing electrocardiogram and ballistocardiogram data.

### BACKGROUND

Numerous types of malfunctions and abnormalities that commonly occur in the cardiovascular system, if not diagnosed and appropriately treated or remedied, will progressively decrease the body's ability to supply sufficient oxygen to satisfy the coronary oxygen demand when the individual encounters stress. The progressive decline in the cardiovascular system's ability to supply oxygen under stress conditions will ultimately culminate in a heart attack, i.e., myocardial infarction event that is caused by the interruption of blood flow through the heart resulting in oxygen starvation of the heart muscle tissue (i.e., myocardium). In serious cases, the consequences are mortality while in less serious cases, permanent damage will occur to the cells comprising the myocardium that will subsequently predispose the individual's susceptibility to additional myocardial infarction events.

In addition to potential malfunctions and abnormalities associated with the heart muscle and valve tissues (e.g., hypertrophy), the decreased supply of blood flow and oxygen supply to the heart are often secondary symptoms of debilitation and/or deterioration of the blood flow and supply system caused by physical and biochemical stresses. While some of these stresses are unavoidable, e.g., increasing age, heredity and gender, many of the causative factors of cardiovascular diseases and malfunction are manageable, modifiable and treatable if their debilitating effects on the cardiovascular system are detected early enough. Examples of such modifiable risk factors include high blood pressure, management of blood cholesterol levels, Diabetes mellitus, physical inactivity, obesity, stress, and smoking. Examples of cardiovascular diseases that are directly affected by these types of stresses include atherosclerosis, coronary artery disease, peripheral vascular disease and peripheral artery disease.

In many patients, the first symptom of ischemic heart disease (IHD) is myocardial infarction or sudden death, with no preceding chest pain as a warning. Screening tests are of particular importance for patients with risk factors for IHD. Coronary angiography is an invasive test that produces angiographic images, which reveal the extent and severity of all coronary arterial blockages and details of the heart musculature. Although coronary angiography is an effective technique, the procedure is invasive and requires the use of local anaesthesia and intravenous sedation.

The most common non-invasive initial screening test for IHD is to measure the electrical activity over a period of time which is reproduced as a repeating wave pattern, commonly referred to as an electrocardiograph (ECG), showing the rhythmic depolarization and repolarization of the heart muscles. Another non-invasive screening test for IHD is ballistocardiography (BCG), which is a method of graphically recording minute movements on an individual's body surface as a consequence of the ballistic i.e., seismic forces associated with cardiac function. These minute movements are amplified and translated by a pick-up device, such as an accelerometer, that is placed onto a patient's sternum, into signals that are recorded on moving chart paper.

Analysis of the various waves and normal vectors associated with electrical and mechanical activity of the heart provided by ECG and BCG waveforms, respectively, yields important diagnostic information. FIGS. 1(a) and 1(b) show the relationship between rhythmic electrical functions and related physical motions of a heart in which FIG. 1(a) is a sample ECG waveform and FIG. 1(b) is a sample BCG waveform.

In order to better understand the ECG and BCG waveforms, an explanation of basic heart function is provided. The heart includes four chambers, the right atrium interconnected with the right ventrical by the tricuspid valve, and the left atrium interconnected with the left ventricle by the mitral valve. Blood is delivered into the right atrium from the upper half of the body via the superior vena cava, and from the lower half of the body via the inferior vena cava. The tricuspid valve is opened by concurrent contraction of the right atrium myocardium and the right ventricular papillary muscles thereby allowing blood flow from the right atrium into the right ventricle, and then closes when the papillary muscles relax. When the myocardium of the right ventricle contracts, blood is forced from the right ventricle through the pulmonary valve into the pulmonary artery which delivers the blood into the lungs wherein it is oxygenated. The oxygenated blood is then returned into the left atrium via pulmonary veins. The oxygenated blood flows from the left atrium into the left ventricle when the mitral valve is opened by concurrent contraction of the left atrium myocardium and the left ventricular papillary muscles thereby allowing blood flow from the left atrium into the left ventricle, and then closed when the papillary muscles relax. The oxygenated blood is then forced out of the left ventricle through the aortic valve into the aorta which delivers the oxygenated blood throughout the body via the peripheral vascular system.

Every rhythmic ‘beat’ of the heart involves three major stages: atrial systole, ventricular systole and complete cardiac diastole. Electrical systole is the electrical activity that stimulates the muscle tissue of the chambers of the heart to make them contract. Atrial systole is the period of contraction of the heart muscles encompassing the right and left atria. Both atria contract concurrently with the papillary muscle contraction thereby forcing open the tricuspid valve and the mitral valve. Electrical systole begins within the sinoatrial node located in the right atrium just below the opening to the superior vena cava. The conduction electrical depolarization continues to travel in a wave downwards, leftwards and posteriorly through both atria depolarising each atrial muscle cell in turn. It is this propagation of charge that can be seen as the P wave on the ECG. This is closely followed by mechanical contraction of the atria that is detected on the BCG as an impact, which corresponds to the “h” peak of the waveform, and recoil, which corresponds to the “i” valley of the waveform. As the right and left atria begin to contract, there is an initial high velocity flow of blood into the right and left ventricles, which is detectable as the “j” peak on the BCG. Continuing atrial contraction as the tricuspid valve begins to close forces an additional lower velocity flow of blood into the right and left ventricles. The additional flow of blood is called the “atrial kick”, which corresponds to the “a-a¹” wave pattern. After the atria are emptied, the tricuspid and mitral valves close thereby giving rise to the downward “g” wave pattern on the BCG.

Ventricular systole is the contraction of the muscles of the left and right ventricles, and is caused the electrical depolarization of the ventricular myocardia giving rise to the QRS complex in the ECG waveform. The downward Q wave is caused by the downward flow of depolarisation through the septum along a specialized group of cells called “the bundle of His”. The R wave is caused by depolarization of the ventricular muscle tissue, while the S wave is produced by depolarization of the heart tissue between the atria and ventricles. As the depolarization travels down the septum and throughout the ventricular myocardia, the atria and sinoatrial node start to polarise. The closing of the tricuspid and mitral valves mark the beginning of ventricular systole and cause the first part of the “lub-dub” sound made by the heart as it beats. Formally, this sound is known as the “First Heart Tone”. As the electrical depolarization of the ventricular myocardia peaks, the AV septum separating the right and left ventricles contracts causing an impact, which corresponds to the “H” peak on the BCG, and a recoil, which corresponds to the “I” valley on the BCG. The ventricular contraction forces the blood from the right ventricle into the pulmonary artery through the pulmonary valve, and from the left ventricle into the aorta through the aortic valve under very high velocity thereby causing the “J” wave in the BCG. The deceleration of blood flow from the left ventricle into the aorta causes a downward decline in the BCG resulting in the “K” wave. As the left ventricle empties, its pressure falls below the pressure in the aorta and the aortic valve closes. Similarly, as the pressure in the right ventricle falls below the pressure in the pulmonary artery, the pulmonary valve closes. The second part of the “Iub-dub” sound, which is known as the “Second Heart Tone”, is caused by the closure of the pulmonary and aortic valves at the end of ventricular systole thereby giving rise to the upward “L” wave of the BCG. Concurrently with the closing of the pulmonary and aortic valves, the AV septum relaxes and moves upward, and the ventricular myocardia is re-polarized giving rise to the “T” wave in the ECG.

Cardiac diastole, which includes atrial diastole and ventricular diastole, is the period of time when the heart relaxes after contraction in preparation for refilling with circulating blood. Atrial diastole is when the right and left atria are relaxing, while ventricular diastole is when the right and left ventricles are relaxing. During the period of atrial diastole, the right atrium is re-filled by deoxygenated blood while the left atrium is re-filled with oxygenated blood. Re-filling of the atria causes a downward “M” wave in the BCG early in diastole which coincides with repolarization of the bundle of His cells, which is shown as the “U” wave in the ECG. As the right and left atria are filled to their maximum capacities, the reflux of blood against the tricuspid valve and mitral valve cause an upward “N” wave in the BCG.

In general, ECG measurements are not particularly sensitive nor are the data very useful for detecting cardiovascular abnormalities or malfunctions. Further, ECG printouts provide a static record of a patient's cardiovascular function at the time the testing was done, and may not reflect severe underlying heart problems at a time when the patient is not having any symptoms. In addition, many abnormal patterns on an ECG may be non-specific, meaning that they may be observed with a variety of different conditions. They may even be a normal variant and not reflect any abnormality at all.

Analysis of BCG wave patterns is typically performed visually by qualified diagnosticians in order to identify normal and abnormal cardiovascular function. The most common BCG wave pattern classification system is known as the Starr system (Starr et al., 1961, Circulation 23: 714-732) and identifies four categories of cardiovascular function depending on the abnormalities in the measured BCG signals. In class 1, all BCG complexes are normal in contour. In class 2, the majority of the complexes are normal, but one or two of the smaller complexes of each respiratory cycle are abnormal in contour. In class 3, the majority of the complexes are abnormal in contour, usually only a few of the largest complexes of each respiratory cycle remaining normal and in class 4, there is such complete distortion that the waves cannot be identified with confidence. In general, a normal healthy person should belong to Starr class 1, and person belonging to class 3 or 4 has a significant abnormality in one or more components of the cardiovascular system. However, the classification is not exact, as it is done visually and depends on the person making the classification.

Despite the limitations associated with visual analysis of ballistocardiogram waveforms, the use of ballistocardiographs as a diagnostic tool is increasing. A typical apparatus for collecting ballistocardiogram data includes a low-friction table and an accelerometer, which transduces the motion of the entire table caused by the systolic ejection of a heart of a subject lying on the table. Currently, due in part to its large size, the use of this type of apparatus is generally limited to research environments.

A need therefore exists for an improved method and apparatus for acquiring and analyzing data relating to a physiological condition of a subject.

### SUMMARY

There is provided herein a method for locating and marking points on a waveform including: providing data corresponding to electrocardiogram and ballistocardiogram waveforms correlated in time; searching the data to locate points corresponding to cardiac events, a location of each of the points corresponding to cardiac events being defined by a rule set; identifying and storing the points corresponding to cardiac events; and outputting a visual representation including the points corresponding to cardiac events marked on the electrocardiogram and ballistocardiogram waveforms.

There is further provided herein an apparatus for acquiring and analyzing data relating to a physiological condition of a subject, the apparatus comprising: a sensor device for coupling to a subject, the sensor device including a three-axis accelerometer and a pair of conductive strips in communication with electrocardiograph lead circuitry, the sensor device for detecting four analog signals and converting the four analog signals to digital signals, one of the four analog signals being an electrocardiograph signal and three of the four analog signals being ballistocardiograph signals corresponding to each axis of the three axis accelerometer; a computer having a processor for applying a rule set to data corresponding to electrocardiogram and ballistocardiogram waveforms correlated in time, the rule set including parameters for locating points corresponding to cardiac events on the electrocardiogram and ballistocardiogram waveforms, and storing the points corresponding to cardiac events with the data; and an output device for outputting a visual representation including the points corresponding to cardiac events marked on the electrocardiogram and ballistocardiogram waveforms.

### DRAWINGS

The following figures set forth embodiments of the invention in which like reference numerals denote like parts. Embodiments of the invention are illustrated by way of example and not by way of limitation in the accompanying figures.

FIG. 1(a) is an example of an electrocardiogram waveform;

FIG. 1(b) is an example of a ballistocardiogram waveform;

FIG. 2 is a schematic diagram of an apparatus for acquiring and analyzing data relating to a physiological condition of a subject according to an embodiment;

FIG. 3 is a perspective view of a sensor device and a data acquisition component of the apparatus of FIG. 2;

FIG. 4 is an isometric view of a wireless sensor device according to another embodiment;

FIG. 5 is a bottom view of the senor device of FIG. 4;

FIG. 6 is a block diagram of selected components of the sensor device of FIG. 4;

FIG. 7 is a block diagram of an apparatus for acquiring and analyzing data relating to a physiological condition of a subject according to another embodiment;

FIG. 8 is a front view of a portable terminal of the apparatus of FIG. 7;

FIG. 9 is a schematic diagram of an apparatus for acquiring and analyzing data relating to a physiological condition of a subject according to another embodiment;

FIG. 10 is a flowchart depicting a method of operation of an apparatus for acquiring and analyzing data relating to a physiological condition of a subject according to another embodiment;

FIG. 11 is a schematic diagram showing an example of an application of an apparatus for acquiring and analyzing cardiovascular data;

FIG. 12 is an isometric view of the sensor device of FIG. 4 and a double-sided ECG electrode;

FIG. 13 is an example of a synchronized electrocardiogram and ballistocardiogram waveform pair captured using an apparatus for acquiring and analyzing data relating to a physiological condition of a subject;

FIG. 14 is a flowchart depicting a method for locating and marking points on a waveform according to an embodiment;

FIG. 15 is a flowchart depicting another method for locating and marking points on a waveform according to an embodiment;

FIG. 16 is a flowchart depicting yet another method for locating and marking points on a waveform according to an embodiment; and

FIG. 17 is a flowchart depicting still another method for locating and marking points on a waveform according to an embodiment.

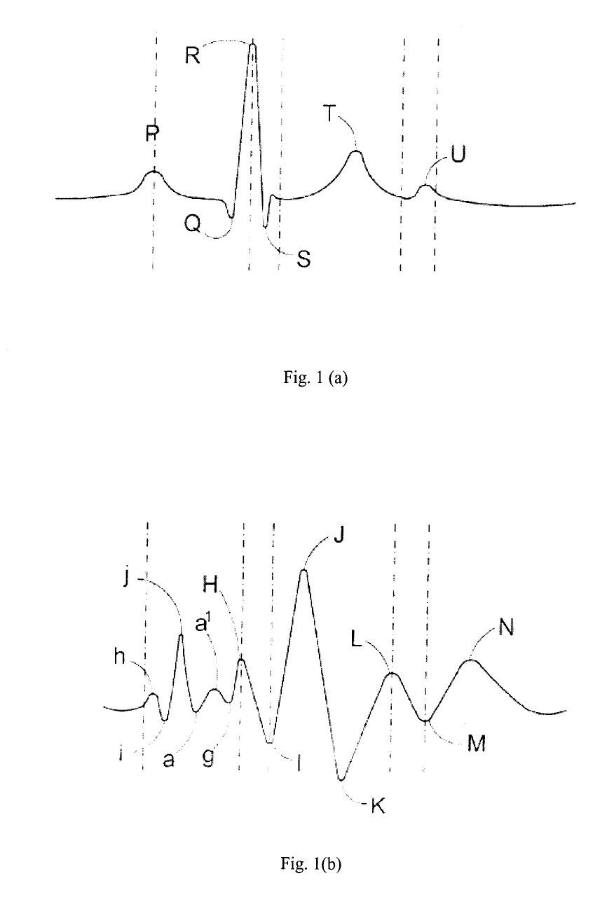

### DETAILED DESCRIPTION OF EMBODIMENTS OF THE INVENTION

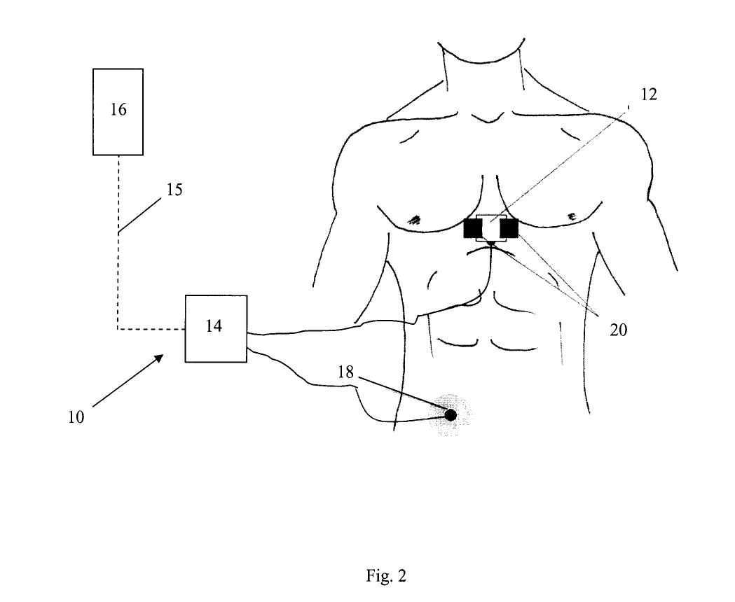

Referring to FIG. 2, an apparatus 10 for acquiring and analyzing data relating to a physiological condition of a subject is generally shown. The apparatus 10 includes a sensor device 12 for coupling to the subject, a data acquisition component 14 and a computer 16. The sensor device 12 is provided to detect four separate analog signals and transmit the analog signals to the data acquisition component 14, one of the four analog signals being an electrocardiograph (ECG) signal and three of the four analog signals being ballistocardiograph (BCG) signals.

The data acquisition component 14 includes a radio device, a power supply and an analog to digital converter, which converts analog signals received from the sensor device 12 into digital signals. The data acquisition component 14 communicates with computer 16 using the radio device. Wireless communication occurs via Bluetooth TM as indicated by dashed line 15. The data acquisition component 14 may alternatively communicate with the computer 16 using another type of wireless technology or via a cable.

The computer is provided to receive the digital signals from the data acquisition component 14. The computer 16 includes a processor for executing software that is stored in computer memory. The software is provided to analyze the digital ECG and BCG signals received from the data acquisition component 14 and output a report relating to the physiological condition of the subject. The report may be printed by a printer (not shown) that is in communication with the computer 16 or, alternatively, the report may be displayed on a display screen (not shown) of the computer 16.

A reference lead 18 is provided to improve the quality of the ECG signal. The reference lead 18 is optional and is used when there is a significant amount of noise affecting the ECG signal. The reference lead 18 is shown coupled to the right side of the subject, however, may alternatively be coupled to another part of the body.

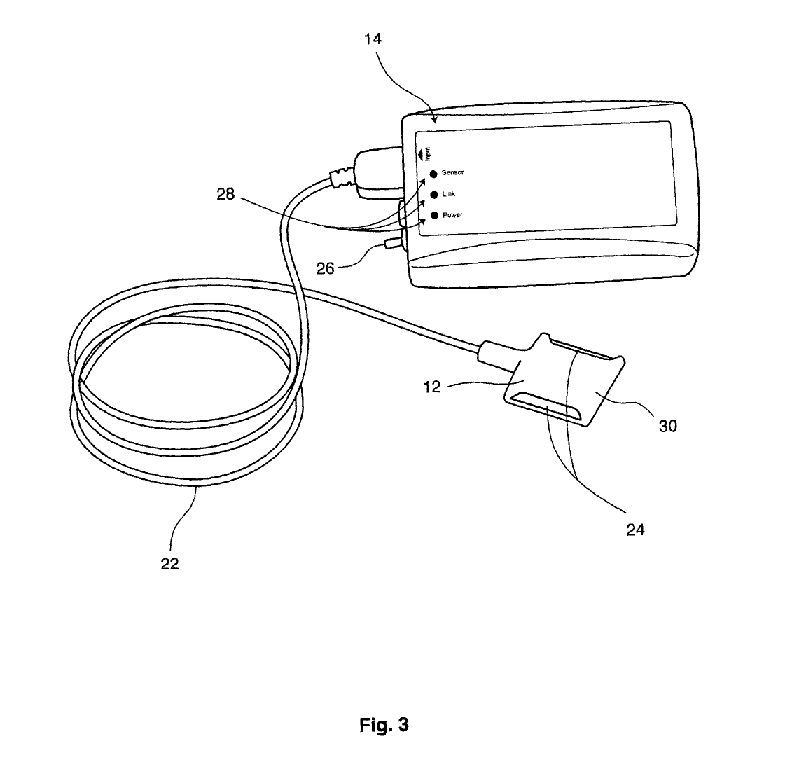

Referring also to FIG. 3, the sensor device 12 and data acquisition component 14 are connected by a cable 22. The sensor device 12 includes a housing 30 in which a pair of conductive strips 24 for detecting the ECG signal and a three-axis accelerometer (not shown) for detecting the BCG signals are provided.

In use, the sensor device 12 is coupled to a sternum of the subject in the orientation shown in FIG. 2 such that the x-axis of the accelerometer extends in the positive direction from head to toe of a subject, the y-axis of the accelerometer extends in the positive direction from right shoulder to left shoulder of the subject and the z-axis of the accelerometer extends in the positive direction from spine to sternum of the subject, in order to obtain BCG signals in the x, y and z directions. Electrode adhesives 20 are coupled between the subject and the sensor device 12 in order to allow for detection of the ECG signal from the subject. A power switch 26 is provided on the data acquisition device 14 and LEDs (light emitting diodes) 28 provide status information relating to power, sensor detection activity and the wireless connection with the computer 16.

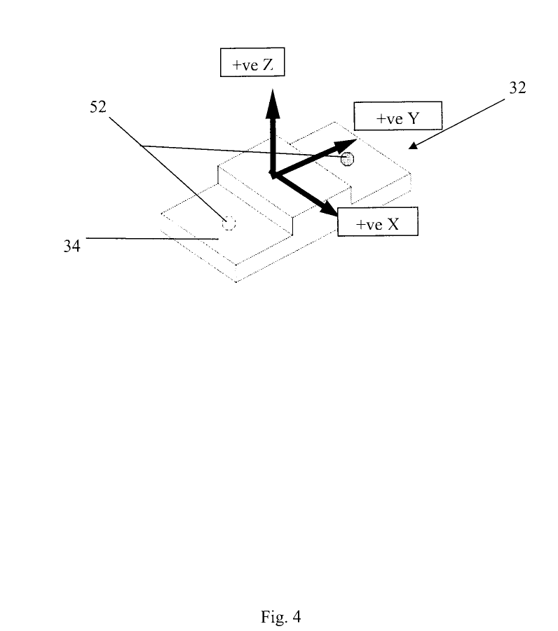

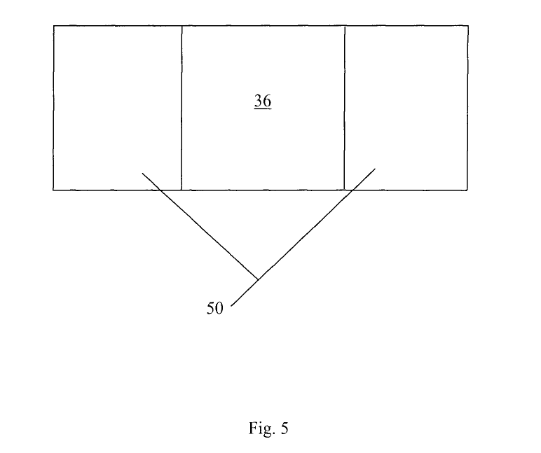

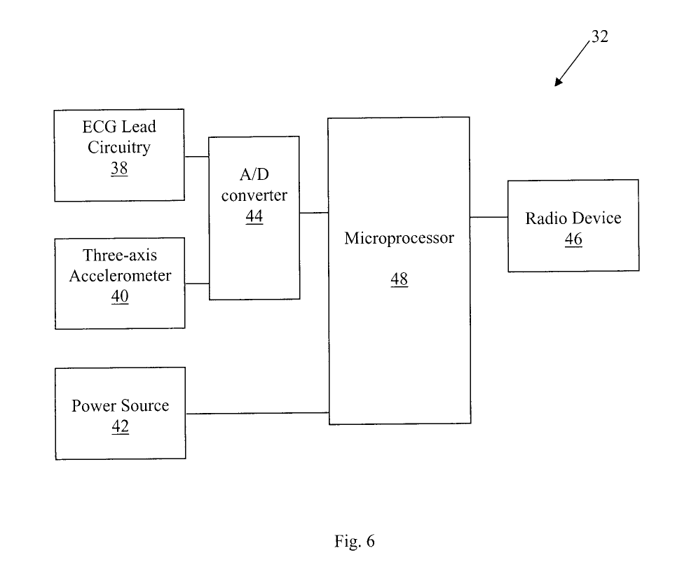

Referring to FIGS. 4 and 5, another embodiment of a sensor device 32 is generally shown. The sensor device 32 of this embodiment is capable of wireless communication and includes the functionality of the sensor device 12 and the data acquisition component 14 of the previous embodiment. Referring also to FIG. 6, the sensor device 32 is provided for use in an apparatus for acquiring and analyzing data relating to a physiological condition of a subject and includes: a housing 34 having a contact surface 36 for coupling to a subject, a three-axis accelerometer 40 that is provided in the housing 34 for sensing vibrations of a chest wall of the subject, conductive strips 50 provided in the contact surface 36 of housing 34 and in communication with electrocardiograph lead circuitry 38 for sensing electrical activity associated with mechanical motion of the heart, an analog to digital converter 44 provided in the housing in communication with the three-axis accelerometer 40 and the electrocardiograph lead circuitry 38 to receive four separate analog signals, one of the four analog signals being an electrocardiograph signal and three of the four analog signals being ballistocardiograph signals corresponding to each axis of the three-axis accelerometer, the analog to digital converter 44 for converting the four separate analog signals into digital signals, a power source 42 provided in the housing and a radio device 46 provided in the housing 34 for transmitting the digital signals to a computer.

The contact surface 36 of the sensor device 32 is provided for coupling to a subject's chest proximal to the sternum. The housing 34 is sized to receive and protect the components of the sensor device 32, while still being small enough for mounting on a subject's chest. The ECG lead circuitry 38, three-axis accelerometer 40, power supply 42, analog-to-digital converter 44, radio device 46 and microprocessor 48, which are mounted in housing 34, provide the sensor device 32 with signal detection, conversion and transmission capabilities. The housing is made of a biocompatible material such as plastic, for example. The housing may alternatively be made of composite or another suitable material.

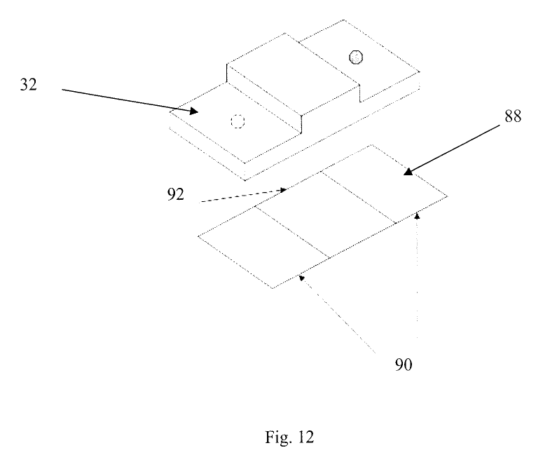

Conductive strips 50, which are shown in FIG. 5, are located at opposite ends of the contact surface 36 and are generally flush therewith. The portion of the contact surface 36 that is located between the conductive strips 50 insulates the strips 50 from one another. The conductive strips 50 detect the ECG signal through electrode adhesives (not shown), which are provided between the conductive strips 50 and the subject's chest. Two separate electrode adhesives may be used or, alternatively, a single electrode adhesive 92, which is shown in FIG. 12, may also be used.

The three-axis accelerometer 40 senses the mechanical motion of the chest wall caused by heart movement in three axes: x, y and z and outputs three separate BCG signals that correspond to the x, y and z axes. Each of these axes, when correlated in time to the Q-wave of an electrocardiogram waveform, provide relevant clinical information about the physical condition of the heart and the circulatory system. An example of a three-axis accelerometer that is suitable for use in the sensor device 32 is a LIS3L02AL MEMS Inertial sensor, which is manufactured by ST Microelectronics.

The sensor device 32 further includes a non-volatile memory (not shown) that is programmed with accelerometer calibration data. Calibration of the three-axis accelerometer occurs at the time of manufacture of the sensor device 32 and is typically performed with the aid of a shake table.

The power source 42 is generally a battery capable of providing sufficient power to operate the sensor device 32. The power source 42 may have a finite life, or alternatively, may be rechargeable.

The analog-to-digital converter 44 is provided in communication with the ECG lead circuitry 38 and accelerometer 40 to receive four separate analog signals: one ECG signal and three BCG signals. The ECG and BCG signals are amplified by amplifiers set to appropriate gain levels and band-limited by linear filtering prior to being sampled by the analog-to-digital converter 44. Any suitable analog-to-digital converter may be used, such as a 12-bit analog-to-digital converter having a sample rate of 500 samples per second, for example.

The radio device 46 is provided to transmit the digital signals, which correspond to the four separate ECG and BCG signals. The radio device 46 may be any device that is capable of wireless communication. In one embodiment, the radio device 28 is a Bluetooth™ communication device capable of short range wireless communication.

The microprocessor 48 communicates with each of the electronic components of the sensor device 32 and generally controls operation thereof.

As shown, the sensor device 32 of FIG. 4 further includes visual indicators 52, which are provided in the sensor device housing 34. The visual indicators are LEDs that display the status of the battery and the wireless link. It will be appreciated by a person skilled in the art that the visual indicators are optional and do not affect operation of the sensor device 32.

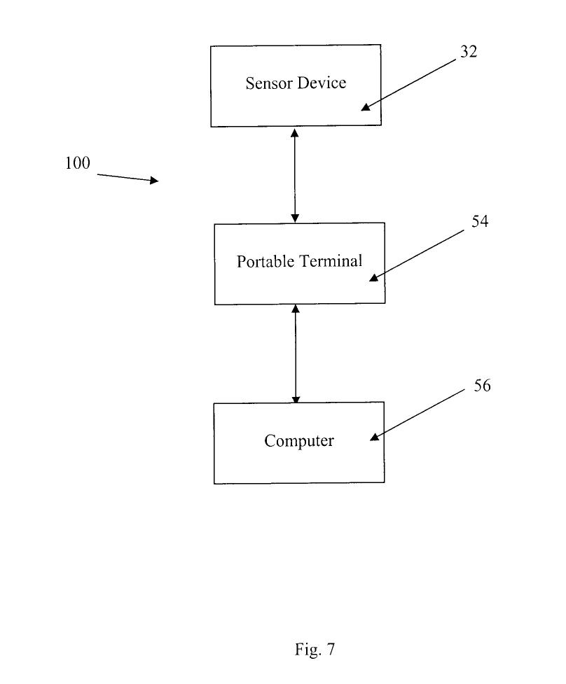

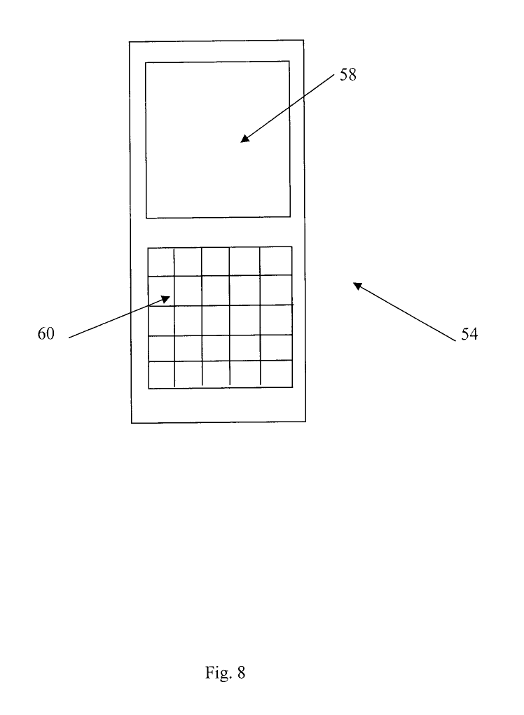

Referring to FIG. 7, another embodiment of an apparatus 100 for acquiring and analyzing data relating to a physiological condition of a subject is generally shown. The apparatus 100 includes the sensor device 32 of FIG. 4, a portable terminal 54 and a computer 56. The portable terminal 54 is provided in communication with the sensor device 32 and the computer 56. As shown in FIG. 8, the portable terminal 54 includes a display screen 58, a keyboard 60, a microprocessor (not shown), a first radio device (not shown) and a second radio device (not shown). The display screen 58 and keyboard 60 provide a user interface that allows an operator of the apparatus 100 to interact with the portable terminal 54.

The portable terminal 54 controls the sensor device 32 by sending commands via the first radio device in order to initiate and terminate detection and transmission of the ECG and BCG signals. The commands are received by the radio device 46 of the sensor device 32 and then executed by the microprocessor 48. The second radio device transmits the digital signals that are received by the portable terminal 54 to the computer 56, which is located remotely. The computer 56 includes software that is stored in memory and is executable by the processor to analyze the digital signals received from the portable terminal 54. The computer 56 further generates and outputs a report relating to the physiological condition of the subject.

For each test that is performed and for which data is sent to the computer 56, an electronic identification number is associated with the data to ensure that the resulting report is associated with the correct subject. It is possible to customize the electronic identification number using the user interface of the portable terminal 54. For example, an operator of the apparatus 100 may input a subject name or a subject identification number using the display screen 58 and keyboard 60. The customized identification information is then electronically linked to the data.

The first radio device of the portable terminal 54 may be any communication device that is capable of short range wireless communication, such as a Bluetooth™ communication device, for example. The second radio device may be any device that is capable of wireless communication. In one embodiment, the second radio device is a wireless network card that communicates with a wireless local area network. In another embodiment, the portable terminal 54 includes a single radio device that is used for communication with both the sensor device 32 and the computer 56.

It will be appreciated by a person skilled in that art that the portable terminal 54 may be any portable terminal that is capable of controlling signal capture from the sensor device 32 and transmitting data received from the sensor device 32 to a computer 56. Suitable commercially available units include those used in event ticketing systems, stock inventory systems, wedding registry systems and other such applications. In addition, the portable terminal 54 is not limited to including the type of user interface that is shown in FIG. 8. The portable terminal 54 may include any suitable type of user interface, such as a touch screen, or a voice recognition system, for example.

In another embodiment, multiple sensor device 32 and portable terminal 54 combinations are deployed at different locations and a single computer 56, which is operated by a third party, receives data from each location. In this embodiment, subject data from different locations is analyzed using computer 56 and the corresponding reports that are generated for each test are sent to the respective portable terminals 54 where the reports may be output on the display 58 or by using a printer. Because the computer 56 includes subject data from different sources, any customized identification information that is associated with the data is stripped prior to the data being sent to the computer 56 in order to maintain subject confidentiality. Following the analysis, the customized identification information is re-attached when the report is received by the portable terminal 54.

It will be appreciated by a person skilled in the art that the number of portable terminals 54 that may be in communication with the computer 56 at any one time is determined by the bandwidth and addressing space. Therefore, multiple sensor device 32 and portable terminal 54 combinations may be deployed at each site.

In another embodiment, the portable terminal 54 includes an electronic code reader, such as a bar code scanner or a radio frequency identification (RFID) reader, for example. Rather than manual entry or selection of a subject name from a database, the electronic code reader would allow the technician to scan an ID bracelet of a patient at a hospital so that the captured ECG and BCG data is automatically associated with the subject.

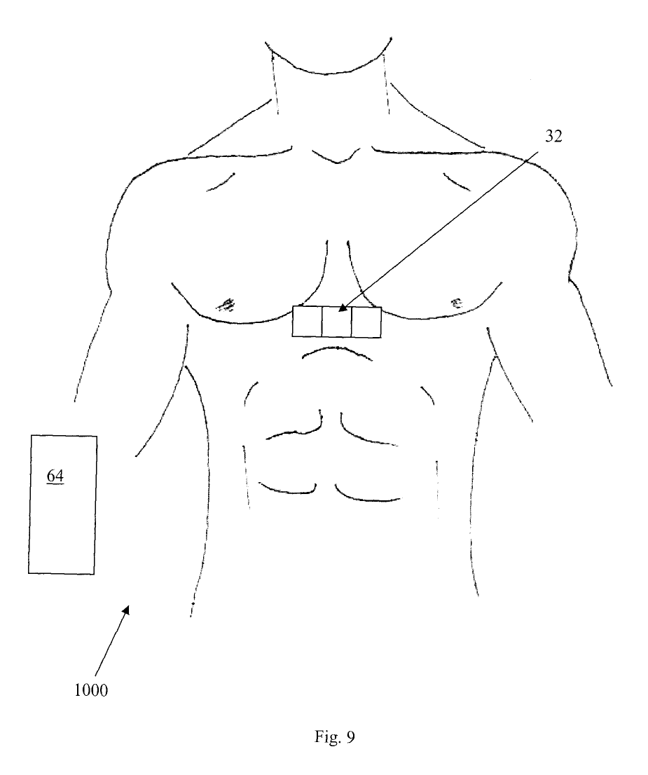

Referring to FIG. 9, still another embodiment of an apparatus 1000 for acquiring and analyzing data relating to a physiological condition of a subject is generally shown. The apparatus 1000 includes a sensor device for coupling to a subject and a computer including a processor that is in communication with the sensor device. The sensor device is provided for detecting, converting and transmitting digital signals corresponding to four analog signals, one of the four analog signals being an electrocardiograph signal and three of the four analog signals being ballistocardiograph signals. The computer is provided for receiving the digital signals from the sensor device and analyzing the digital signals. The computer further generates and outputs a report relating to the physiological condition of the subject.

As shown in FIG. 9, the apparatus 1000 includes the sensor device 32 of FIGS. 4 to 6 and a portable terminal 64. The portable terminal 64 incorporates all of the functionality of the portable terminal 54 and computer 56 of the embodiment of FIG. 7. The portable terminal 64 includes a radio device (not shown), a user interface (not shown), a microprocessor (not shown) and a computer memory (not shown) that stores software that is executable by the microprocessor.

The portable terminal 64 controls the sensor device 32 by sending commands wirelessly via the radio device in order to initiate and terminate detection and transmission of the ECG and BCG signals. The portable terminal 64 receives the digital ECG and BCG signals, analyzes the signals and outputs a report relating to the physiological condition of the subject.

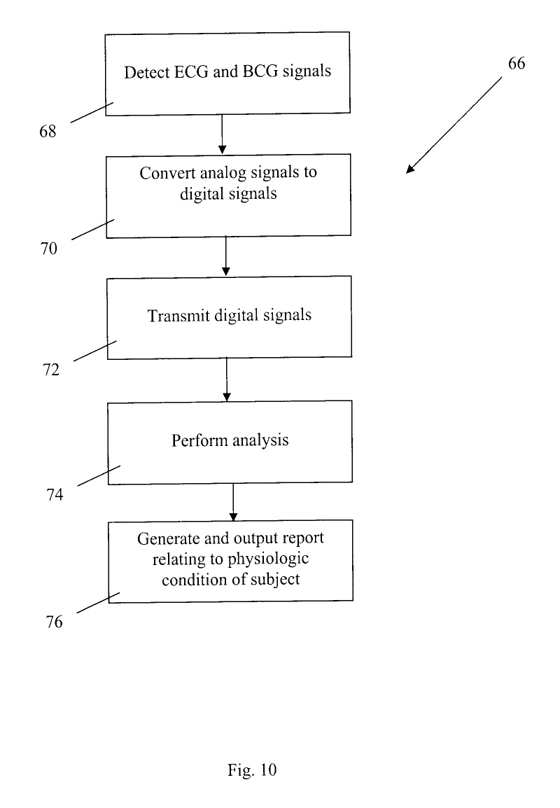

Operation of the apparatus' 10, 100 and 1000 will now be described with reference to FIG. 10, which shows a method 66 for acquiring and analyzing data relating to a physiological condition of a subject. The method is executed once for each test that is performed on a subject. At step 68, the ECG and BCG signals are detected by the sensor device. In order to detect the signals, conductive hydrogel electrode adhesives are applied to the subject's chest across the sternum and the sensor device is coupled thereto. The adhesion provided by the electrodes is sufficient to maintain for the sensor device in position for at least the duration of the test. When coupled to the chest, the sensor device is oriented such that the x-axis of the accelerometer extends in the positive direction from head to toe of a subject, the y-axis of the accelerometer extends in the positive direction from right shoulder to left shoulder of the subject and the z-axis of the accelerometer extends in the positive direction from spine to sternum of the subject. The orientation of the x, y and z axes relative to the sensor device is shown in FIG. 4. Detection of the signals is initiated by a ‘start’ command that is received by the sensor device and detection continues until an ‘end’ command is received. The command may be issued by pressing a designated key on the computer or portable terminal that is in communication with the sensor device. The same key, or a different key, is then pressed in order to send a “stop” command to the sensor device upon completion of the test.

As the signals are detected, they are amplified and converted to digital signals in real time, as indicated at step 70. Once converted, the digital signals are transmitted to the computer, as indicated at step 72. The transmission may occur via the portable terminal or may be direct from the sensor device to the computer. Once the digital signals are received by the computer, an analysis of the BCG data is performed, as indicated at step 74. At step 76, a report relating to the physiological condition of a subject is generated and output by the computer.

The report that is generated by the computer 16 may take a number of different forms depending on the particular application. The reports may be customized to provide only the information that is desired for each application. The report may be printed or displayed by the computer or printed or displayed by the portable terminal. Other methods for outputting the report may also be provided.

In another embodiment, signal detection is initiated by a ‘start’ command that includes a test duration time. In operation, the sensor device begins detecting signals upon receiving the ‘start’ command and continues detecting the signals until the test duration time has elapsed. The sensor device stops detecting signals once the duration time has elapsed without receiving an ‘end’ command. The test duration time may be manually input by the operator or may default to a predetermined time. The test duration time for a typical test is between 10 and 60 seconds, however, longer tests are also possible.

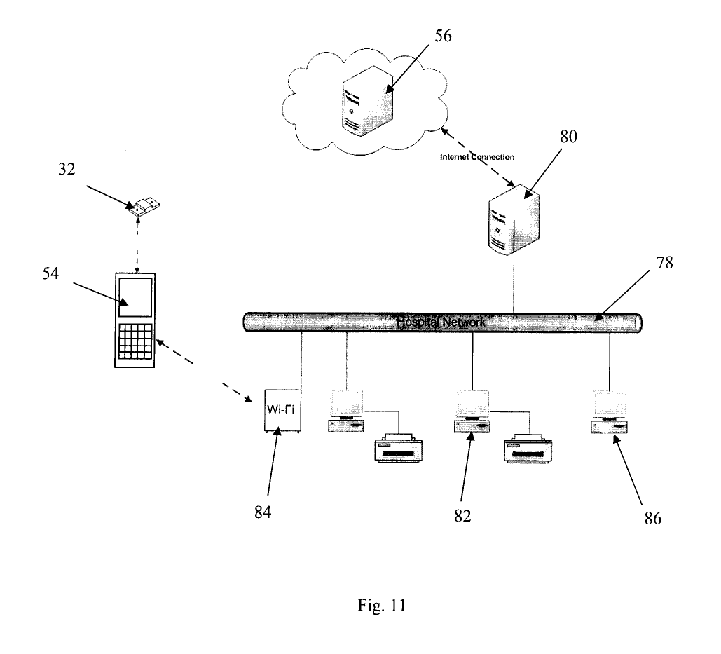

Referring to FIG. 11, an application of apparatus 100 is generally shown. In this application, the apparatus 100 is configured for use in a hospital environment. The apparatus 100 is provided in communication with a local area network (LAN) 78 of the hospital so that data acquired using the apparatus 100 may be linked to patient records that are stored in a Patient Management and Reporting System (PMR) computer 80 on the LAN 78. Reports generated by the apparatus 100 and other patient information is accessible by hospital staff by using a plurality of user stations 82, which communicate with the PMR computer 80 over the LAN 78. Each user station 82 includes a display screen and a printer to view and print patient records.

In operation, a patient is prepared for a test by applying electrode adhesives to the patient's sternum and coupling the sensor device 32 to the electrode adhesives. Prior to the initiation of data collection, an operator of the apparatus 100 inputs patient identification (ID) information into the portable terminal 54. The patient ID input may be entered via the keyboard or by reading an electronic ID from a patient bracelet, for example. Once the patient ID has been determined, the operator sends a ‘start’ command to the sensor device 32. The command may be issued by pressing a designated key on the portable terminal 54, for example. In response to the “start” command, digital signal data is streamed to the portable terminal 54. The same key, or a different key, is then pressed in order to send a “stop” command to the sensor device 32 upon completion of the test. Alternatively, the original ‘start’ command may include a test duration time so that the signal detection automatically stops once the test duration time has been reached.

During the data collection process, digital signals are transmitted from the sensor device 32 to the portable terminal 54 via Bluetooth™. The portable terminal 54 electronically associates the digital signals with the patient ID and then transmits the digital signals to the PMR computer 80 via a wireless access point 84 to the LAN 78. The PMR computer 80 strips the data of any patient information and then sends the data to the computer 56 over the internet using a secure data transfer protocol.

ECG and BCG signal data, which corresponds to synchronized ECG and BCG waveforms, is received by the computer 56 and the computer processor performs an analysis using software that is stored on the computer 56. Following analysis, a report is produced and forwarded to the PMR computer 80 of the hospital. The report is stored on the PMR computer 80 in the appropriate patient record.

In one example, the apparatus 100 is used in a hospital emergency room (ER) to determine the effect of medication on specific cardiac events. The sensor device 32 is applied upon initial admission of a suspected cardiac patient to the ER and a preliminary analysis is performed. Following medication, subsequent analysis is performed to determine the effects on, for instance, the timing of the closing of the mitral valve. An advantage of analyzing the BCG data is that changes may be seen earlier in the mechanical motion of the heart than in the related electrical activity.

An analysis suite 86, which allows for manual analysis of raw electrocardiogram and ballistocardiogram signal data that is acquired using the sensor device 32, is also shown in FIG. 11. The analysis suite 86 is operable on a computer that includes a display screen. The analysis suite 86 is optional and allows doctors or technicians to view patient electrocardiograms and ballistocardiograms that may be generated using the raw data rather than receiving report output.

It will be appreciated by a person skilled in the art that ECG and BCG signal data and report data may be managed in many different ways. In the example of FIG. 11, the ECG and BCG signal data is forwarded from the sensor device 32 to the portable terminal 54 to the PMR computer 80 and on to the computer 56, where the data is analyzed. The report is generated by the computer 56 and then sent to the PMR computer 80, where it is stored. In another embodiment, the ECG and BCG signal data is stored and transmitted in a file. The file may be generated by either the portable terminal 54 or PMR computer 80 and the ECG and BCG signal data may be sent to the computer 56 in the file or, alternatively, the file may be opened and the raw ECG and BCG signal data may be transmitted. In yet another embodiment, the file is generated by the portable terminal 54 and written to a drive of the PMR computer 56. A message is sent to the PMR computer 80 to advise that the file has been stored thereon.

An advantage of the apparatus' described herein is that the operator does not need to be a qualified diagnostician. The operator may be a nurse, a technician, a doctor or another hospital employee who received the minimal training required to use the apparatus'. Another advantage is that the acquisition, analysis and reporting of the physiological condition occurs in a short period of time so that a greater number of subjects may be tested in a shorter period of time.

Referring to FIG. 12, a double-sided electrode adhesive 88 for use with the sensor device 32 is generally shown. The double-sided electrode adhesive 88 includes a pair of electrocardiograph electrodes 90 that are spaced apart. An insulating portion 92 is provided between the electrodes 90. Each side of the double-sided electrode adhesive 88 is sticky so that it may be sandwiched between the subject's chest and the contact surface 36 of the sensor device 32 to couple the sensor device 32 to the subject's chest. The double-sided adhesive 88 is typically used for a single test, which aids in sterility.

In use, the double-sided electrode adhesive 88 is first adhered to a subject's chest. The sensor device 32 is then aligned with the double-sided electrode adhesive 88 and adhered thereto. When in position, the conductive strips 50 of the sensor device 32 are in contact with the electrodes 90 of the double-sided electrode adhesive 88 to allow for detection of ECG signals. Once the sensor device 32 is in position, the apparatus 100, 1000 including the sensor device 32 operates in a manner that has been previously described. The adhesive properties of the double-sided electrode adhesive 88 maintain the sensor device 12 in position on the subject for at least the duration of the test.

It will be appreciated by a person skilled in the art that rather than first adhering the double-sided electrode adhesive 88 to the subject, the double-sided electrode adhesive 88 may be first adhered to the sensor device 32. The double-sided electrode adhesive 88 with the sensor device 32 coupled thereto may then be adhered to the subject's chest.

As has been described, apparatus' for acquiring and analyzing data relating to a physiological condition of a subject includes at least a sensor device and a computer including software for analyzing the digital signals that are output from the sensor device. Methods for analyzing the digital signals will now be described.

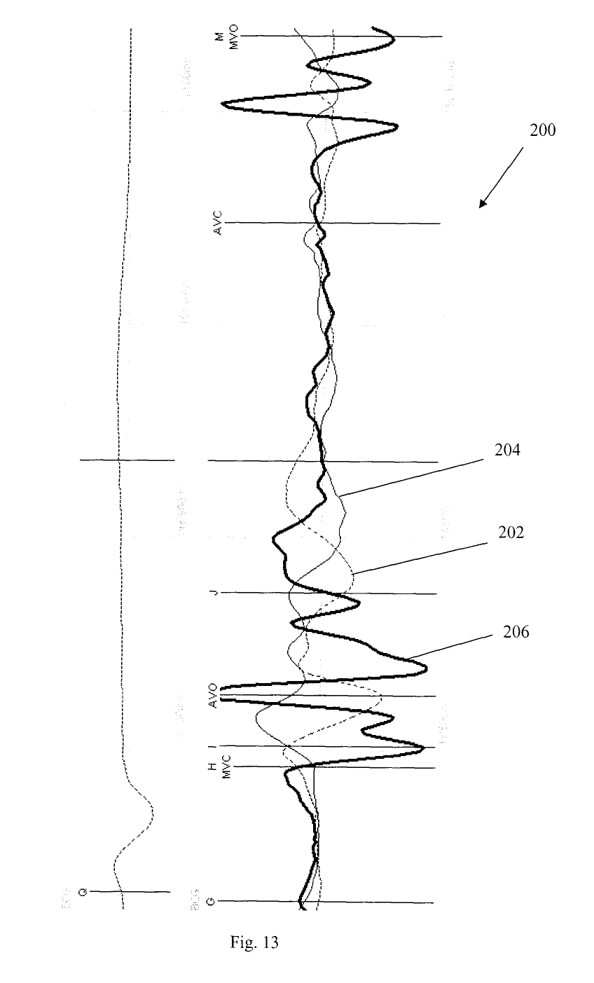

An example of a synchronized electrocardiogram-ballistocardiogram (ECG-BCG) waveform set 200 is shown in FIG. 13. The ECG-BCG waveform set is a visual representation of captured ECG and BCG signal data. The ECG-BCG waveform set is automatically synchronized in time because detection of the ECG and BCG signals by the sensor device begins simultaneously in response to the ‘start’ command. As shown, the ballistocardiogram includes three separate waveforms that correspond to the different axes of the accelerometer. The waveforms are identified as follows: the x-axis waveform 202 is shown as a dotted line, the y-axis waveform 204 is shown as a thin line and the z-axis waveform 206 is shown as a thick line.

In order to correlate the ECG and BCG signals detected by the sensor device with heart activity of a subject, each heartbeat of the captured, synchronized ECG-BCG waveform set is annotated with a plurality of different cardiac events. As will be appreciated by a person skilled in the art of electrocardiography and ballistocardiography, the term “annotation” is commonly used to refer to a mark that is provided on a waveform to identify a cardiac event.

As shown in FIG. 13, some of the different cardiac events are identified using the reference letters: Q, G, H/MVC, I, J, AVO, AVC and M/MVO. The Q annotation denotes depolarization of the inter-ventricular septum; the G annotation denotes atrial contraction; the H annotation denotes the mitral valve close event; the I annotation denotes isovolumic movement; the J annotation denotes the rapid ejection period; the AVO annotation denotes the aortic valve open event; the AVC annotation denotes the aortic valve close event and the M annotation denotes the mitral valve open event.

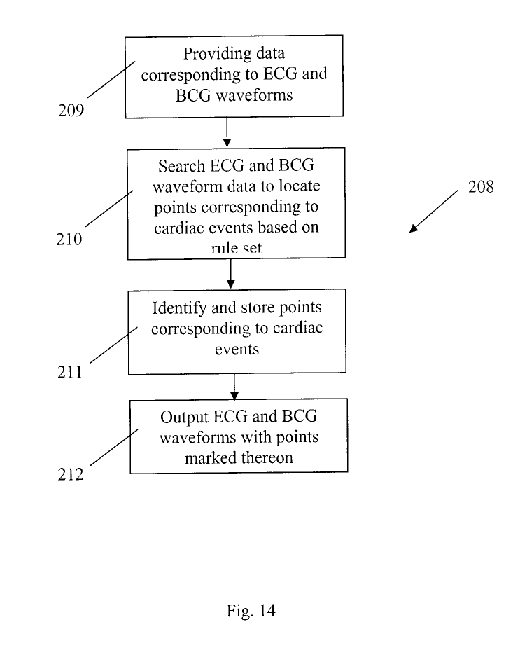

Referring to FIG. 14, a method for locating and marking points on a waveform 208 is provided. The method is a post-processing method that is performed on a synchronized ECG-BCG waveform set that has been captured using one of the apparatus' for acquiring and analyzing data relating to a physiological condition of a subject disclosed herein. The method includes: at step 209, providing data corresponding to electrocardiogram and ballistocardiogram waveforms correlated in time, at step 210, searching the data to locate points corresponding to cardiac events, a location of each of the points corresponding to cardiac events being defined by a rule set, at step 211, identifying and storing the points corresponding to cardiac events and, at step 212, outputting a visual representation including the points corresponding to cardiac events marked on the electrocardiogram and ballistocardiogram waveforms.

The points corresponding to cardiac events and data are stored in computer memory during application of the method of FIG. 14. Following the analysis, a computer-readable file is generated including the points corresponding to cardiac events and the data that is stored in the computer memory. The computer-readable file may be automatically generated or, alternatively, the operator may be provided with an option to: (i) store the analyzed test data in a computer-readable file or (ii) discard the analyzed test data. In addition, the computer-readable file may be generated prior to the method of FIG. 14 being applied. In this embodiment, the computer-readable file, which includes the data corresponding to the electrocardiogram and ballistocardiogram waveforms, is searched. When the analysis is complete, the computer-readable file is rewritten including test data and points corresponding to cardiac events.

The rule set includes rules governing the location of each cardiac event on the electrocardiogram and ballistocardiogram waveforms. The rules are applicable to digital ECG and BCG signals that have been normalized to ratios corresponding to 60 beats per minute. The rules are structured based on the following parameters, which can be better understood with reference back to FIG. 13.

The Q annotation is located where the waveform first deflects in an upward or downward direction and is followed by a local peak or a local valley depending on the direction of deflection. The local peak or valley occurs within 100 ms.

The G annotation is the highest peak on the BCG z-Axis within ±20 ms of the Q Annotation.

The H/MVC annotation is located within 50 ms±20 ms of the Q annotation where: the BCG z-Axis and the BCG x-axis cross and the BCG z-Axis is moving in a downward direction.

The I annotation is the first negative valley following the H/MVC annotation.

The AVO annotation occurs within 90 ms±40 ms of the Q annotation and is the first positive peak following the H/MVC annotation.

The J annotation occurs within 170 ms±40 ms of the Q annotation and is located where the BCG z-axis and the BCG x-axis cross and the BCG z-axis is moving in an upward direction.

The AVC annotation occurs within 400 ms±100 ms of the Q annotation and is located where the BCG z-Axis and the BCG x-axis cross.

The M/MVO annotation is denoted as the second or third negative valley following the AVC annotation and occurs within 450 ms±100 ms. If the waveform contains three negative valleys following the AVC Annotation, the M/MVO Annotation is the third negative valley, otherwise it is the second negative valley.

It will be appreciated by a person skilled in the art that the error incorporated into the time windows associated with each of the rules have been established based on trial and error. Thus, the size of the time windows may be increased or decreased.

Once the data has been searched and the rules have been applied thereto, the points corresponding to cardiac events are stored in association with the respective annotation names. An annotated ECG-BCG waveform set is then output by the computer, as indicated at step 212.

The location of the points corresponding to cardiac events may be stored in many different ways. For example, a value that indexes into the array of data points of the ECG-BCG waveform set may be provided for each annotation name. Alternatively, the annotations may be defined by a number containing at least as many bits as annotations in order to identify which annotations have been marked, followed by an ordered list of indices.

In operation, a test on a subject is performed using the apparatus 10, 100, 1000. Once the sensor device has been coupled to the subject and data capture has been initiated, the sensor device captures and transmits ECG and BCG digital signals corresponding to multiple heart beats wirelessly to the computer. The method 208 of FIG. 14 is then applied to the data by the computer processor in order to locate and mark points corresponding to cardiac events. Once the points have been saved, the annotated ECG-BCG waveform set is output by the computer to a display screen. The annotated ECG-BCG waveform set may then be further analyzed by a qualified doctor or technician in order to evaluate performance characteristics of the heart and identify any abnormalities in cardiac function of the subject.

It will be appreciated by a person skilled in the art that the report may be output to a printer or another output device instead of, or in addition to, being output to a display of the computer.

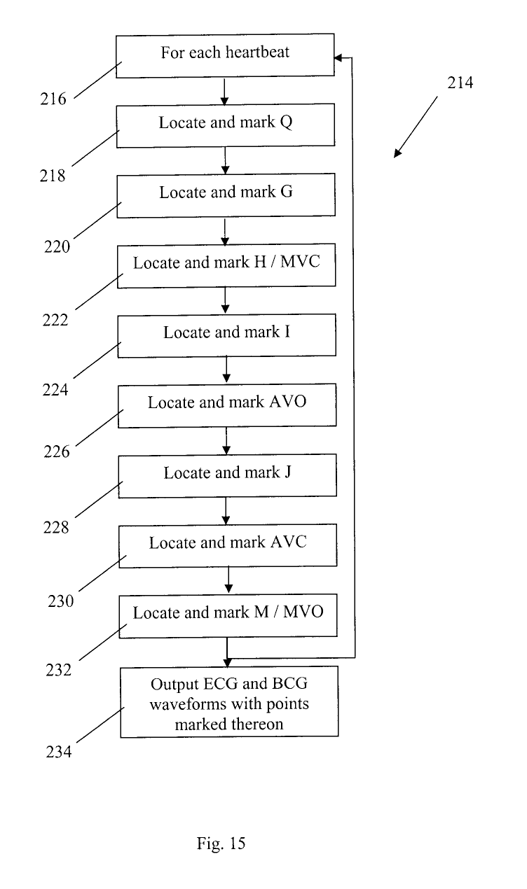

Referring to FIG. 15, another method for locating and marking points on a waveform 214 is provided. This method is similar to the method of FIG. 14, however, is performed on a heart beat by heart beat basis. At steps 216 to 232, ECG-BCG signal data is searched as it is received by the computer in order to locate the cardiac events: Q, G, H/MVC, I, J, AVO, AVC and M/MVO using the rule set previously described in relation to the embodiment of FIG. 14. Once located, the points corresponding to the cardiac events are stored and an annotated ECG-BCG waveform set is output, as indicated at step 234. As indicated by FIG. 15, the points corresponding to cardiac events are located and marked in the order that they occur in time so that each heart beat may be annotated in real time.

Operation of the method 214 is similar to operation of the method 208 of FIG. 14, however, annotated waveforms are displayed following each heart beat. It will be appreciated by a person skilled in the art that the annotated waveforms are provided in “soft real time” rather than real time. A lag exists to account for the time required to receive and process the signals from the sensor device.

The report that is generated and outputted in step 76 of the method of FIG. 10 includes information gathered from the annotated ECG-BCG waveform set. Examples of different types of reports include: an isovolumic contraction time report, which plots the time intervals between MVC and AVO cardiac events, an isovolumic relaxation time report, which plots the time intervals between AVC and MVO cardiac events, and a heart rate report, which plots the heart rate trend of the ECG-BCG waveform set. The report may further include information gathered from different tests performed on the same subject. For example, information from a pre-exercise test may be included in a report with information from a post-exercise test. Similarly, information from a test performed prior to administering a drug to a subject may be included in a report with information from a test performed after administering a drug to the subject. It will be appreciated that the report is not limited to the examples provided herein. The report may include any type of information obtainable from the ECG-BCG waveform set and may be provided in any suitable format. Further, the report may include data from the annotated ECG-BCG waveform set that has been further analyzed using another analysis method.

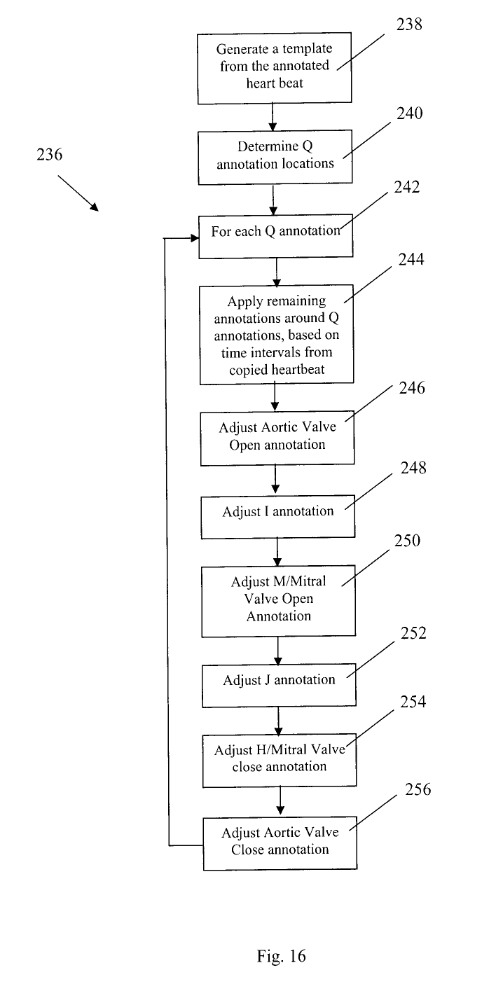

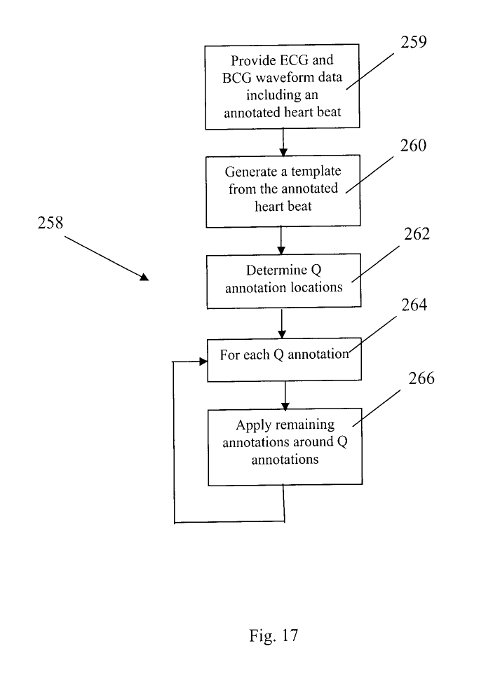

Referring now to FIG. 16, another method for locating and marking points on a waveform 236 is provided. This method is a post-processing method that is performed following manual annotation of a single heart beat of a captured ECG-BCG waveform set. As such, this method and the method of FIG. 17 are used with embodiments that allow for user interaction during data analysis, such as apparatus 10 of FIG. 2, for example. Manual annotation is performed by a technician, who has been trained to visually identify each cardiac event. The manual annotation is performed using an input device, such as a keyboard, or a mouse, for example, that communicates with the computer. The technician identifies points, which correspond to cardiac events, on the electrocardiogram and ballistocardiogram waveforms and the points are stored along with the electrocardiogram and ballistocardiogram waveform data. The test data corresponding to the electrocardiogram and ballistocardiogram waveforms may alternatively be stored in a computer-readable file for annotation and analysis at a later time.

Once an annotated heart beat has been produced, the method of FIG. 16 is initiated. First, a template is generated using the annotated heart beat, as indicated at step 238. The template uses the Q annotation as a reference event and the time interval between the Q annotation and all other annotations referenced in the annotated heart beat are stored for use in extrapolation.

At step 240, Q annotation locations throughout the captured waveform are determined by searching on the electrocardiogram waveform for the location in each heart beat where the waveform first deflects in an upward or downward direction, and is followed by a local peak or a local valley depending on the direction of deflection. This local peak or valley occurs within 100 ms.

A loop is then initiated at step 242. For each Q location, the remaining annotations are determined relative to the Q location based on time intervals from the template, as indicated at step 244. For example, if in the template Q is marked at 10 ms and G is marked at 16 ms, the time difference between these annotations is +6 ms. Therefore, for each Q annotation, a G annotation is marked at the location of the Q annotation plus 6 ms.

Once the annotations have been applied to the waveform, adjustments are then made to optimize the cardiac event locations. The annotations are adjusted to coincide with landmarks that are located within a time window extending on either side of the previously determined reference location. The landmarks for optimizing each cardiac event location may be different and include: lowest point on the ballistocardiogram waveform, highest point on the ballistocardiogram waveform, intersection of two ballistocardiogram waveforms and smallest distance between two ballistocardiogram waveforms.

At step 246, the aortic valve open annotation (AVO) is adjusted. A ±10 ms window within the BCG z-axis waveform on either side of the aortic valve open annotation location that was previously determined at step 244 is searched and the highest point in this window is located. The aortic valve open annotation is then changed to this location.

At step 248, the I annotation is adjusted. A ±10 ms window within the BCG z-axis waveform on either side of the I annotation location that was previously determined at step 244 is searched and the lowest point in this window is located. The I annotation is then changed to this location.

At step 250, the M/mitral valve open location is adjusted. A ±10 ms window within the BCG z-axis waveform on either side of the M/mitral valve open (M/MVO) location that was determined at step 244 is searched and the lowest point in this window is located. The M/mitral valve open annotation is then changed to this location.

At step 252, the J annotation is adjusted. A ±10 ms window on either side of the J location that was previously determined at step 244 is searched and the location where the BCG z-axis and the BCG x-axis cross within this window is determined. The J annotation is then changed to this location. If the waveforms do not cross within this window, the J annotation is changed to the location where the BCG z-axis and the BCG x-axis are closest to one another.

At step 254, the H/mitral valve close (H/MVC) annotation is adjusted. A ±10 ms window on either side of the H/mitral valve close location that was previously determined at step 244 is searched and the location where the BCG z-axis and the BCG x-axis cross within this window is determined. The H/mitral valve close annotation is then changed to this location. If the waveforms do not cross within this window, the H/mitral valve close annotation is changed to the location where the BCG z-axis and the BCG x-axis are closest to one another.

Finally, the aortic valve close annotation (AVC) is adjusted, as indicated at step 256. A ±10 ms window on either side of the aortic valve close location that was previously determined at step 244 is searched and the location where the BCG z-axis and the BCG x-axis cross within this window is determined. The aortic valve close annotation is then changed to this location. If the waveforms do not cross within this window, the aortic valve close location is changed to the location where the BCG z-axis and the BCG x-axis are closest to one another.

In operation, a test on a subject is performed using the apparatus 10. Digital signals corresponding to multiple heart beats are captured and transmitted wirelessly to the computer. When the test is complete, the computer processes the digital signals and outputs a synchronized ECG-BCG waveform set to a display screen of the computer. A technician then analyzes the waveform data and annotates all of the cardiac events for a single heart beat using an input device of the computer. The method of FIG. 16 is then performed by the computer processor to annotate the remaining heart beats of the waveform. An annotated BCG waveform is then output to an output device, such as the display screen of the computer or a printer, for example. The annotated ECG-BCG waveform set may then be further analyzed by a qualified doctor or technician in order to evaluate performance characteristics of the heart and identify any abnormalities in cardiac function of the subject.

It will be appreciated by a person skilled in the art that the ±10 ms time windows associated with each of the optimization steps have been established based on testing of the method. The size of the time windows may be increased or decreased.

Referring to FIG. 17, another method for locating and marking points on a waveform 258 is shown. In this embodiment, the optimization steps 246 through 256 of FIG. 16 are removed and optimization parameters are incorporated into the template as a rule set.

The method includes: at step 259, providing electrocardiogram and ballistocardiogram waveform data correlated in time and extending for at least two heart beats, one of the at least two heart beats being an annotated heart beat having cardiac events identified thereon, the cardiac events including a reference event marked on an electrocardiogram waveform, at step 260, generating a template based on the annotated heart beat, the template including time intervals measured from the reference event to other cardiac events and, at steps 262 to 266, locating the reference event on each non-annotated heart beat and applying the template to determine locations of the other cardiac events.

The template is generated using both the annotated heartbeat and the rule set. For each cardiac event, the time interval from the Q annotation, which is the reference event of this embodiment, is used to locate a ±10 ms window on the waveform. This portion of the waveform is searched based on the optimization parameters and the cardiac event annotation location is determined. For example, for the aortic valve open annotation (AVO), a ±10 ms window is located based on a time interval from the Q annotation then the window is then searched to locate the highest point on the BCG waveform z-axis. The highest point then becomes the AVO annotation location.

The Q annotation locations throughout the captured waveform are determined by locating and marking the point on the electrocardiogram waveform where the waveform first deflects in an upward or downward direction, and is followed by a local peak or a local valley depending on the direction of deflection. This local peak or valley occurs within 100 ms. The remaining annotation locations are then determined relative to the Q locations based on time intervals and rules from the template.

It will be appreciated by a person skilled in the art that one identifiable point on the ECG waveform is required to perform the methods of FIGS. 14 to 17. The rules have been constructed with respect to the Q reference event, which corresponds to depolarization of the inter-ventricular septum. The rules could alternatively be constructed with respect to the R reference event, which corresponds to ventricular activation, on the ECG waveform instead of the Q point. An example of a post-processing method for determining the R locations that may be used along with the method of FIGS. 16 and 17 is presented in “ECG Beat Detection Using Filter Banks” to Afonso et al., published in IEEE Transactions on Biomedical Engineering, Vol. 46, No. 2, February 1999, which is herein incorporated by reference. Other methods that are known in the art may alternatively be used to determine the location of the R reference event in an ECG waveform.

In addition, other cardiac events may be located and marked on the synchronized ECG-BCG waveform set such as early diastole (ED), late diastole (LD), and aortic valve open onset (AVOO), for example.

Using the apparatus' and the methods described herein, it is possible to provide a more timely diagnosis than may be provided using the traditional methods of annotating every heartbeat of a captured, synchronized ECG-BCG waveform set manually. The apparatus' and methods allow for a greater number of subjects to be tested and provided with test results in a shorter period of time.

Specific embodiments have been shown and described herein. However, modifications and variations may occur to those skilled in the art. All such modifications and variations are believed to be within the scope and sphere of the present invention.

1-9. (canceled)

10. An apparatus for acquiring and analyzing data relating to a physiological condition of a subject, said apparatus comprising:
a sensor device for coupling to said subject, said sensor device for detecting four analog signals, one of said four analog signals being an electrocardiograph signal and three of said four analog signals being ballistocardiograph signals, converting said four analog signals to digital signals and transmitting said digital signals; and
a computer including a processor in communication with said sensor device, said computer configured for receiving and analysing data representative of said digital signals and for generating a report relating to said physiological condition of said subject.

11. The apparatus as claimed in claim 1, wherein said computer is further configured for forwarding said report to one or more other computers, and/or for outputting said report to an output device, wherein said output device is a display screen and/or a printer.

12. The apparatus as claimed in claim 10, wherein said sensor device comprises a three-axis accelerometer for detecting said ballistocardiograph signals and wherein each of said ballistocardiograph signals corresponds to an axis of said three axis accelerometer.

13. The apparatus as claimed in claim 10, wherein said sensor device comprises a pair of conductive strips in communication with electrocardiograph lead circuitry for detecting said electrocardiograph signals.

14. The apparatus as claimed in claim 10, wherein said computer is configured for communicating with said sensor device to control detection of said four analog signals.

15. The apparatus as claimed in claim 10, further comprising a portable terminal in communication with said sensor device and said computer, said portable terminal for controlling detection of said four analog signals, for receiving said digital signals from said sensor device and for transmitting data representative of said digital signals to said computer.

16. The apparatus as claimed in claim 15, wherein said portable terminal is configured for receiving subject identification information and associating said subject identification information with said digital signals.

17. The apparatus as claimed in claim 16, wherein said portable terminal includes a device for reading an electronic code, said electronic code including said subject identification information.

18. The apparatus as claimed in claim 10, wherein said computer is a portable terminal.

19. The apparatus as claimed in claim 18, wherein said portable terminal is configured for receiving subject identification information and associating said subject identification information with said digital signals.

20. The apparatus as claimed in claim 19, wherein said portable terminal includes a device for reading an electronic code, said electronic code including said subject identification information.

21. The apparatus as claimed in claim 10, wherein said computer is in communication with said sensor device via a wireless connection.

22. The apparatus as claimed in claim 10, wherein said computer is in communication with said sensor device via a wired connection.

23. The apparatus as claimed in claim 15, wherein said portable terminal is in communication with said sensor device and/or said computer via a wireless connection.

24. The apparatus as claimed in claim 10, wherein the computer is configured for applying a rule set to data corresponding to electrocardiogram and ballistocardiogram waveforms correlated in time, said electrocardiogram and ballistocardiogram waveforms correlated in time generated from said digital signals, said rule set including parameters for locating points corresponding to cardiac events on said electrocardiogram and ballistocardiogram waveforms.

25. The apparatus as claimed in claim 24, wherein said computer is further configured for storing said points corresponding to cardiac events with said data corresponding to electrocardiogram and ballistocardiogram waveforms correlated in time.

26. The apparatus as claimed in claim 24, wherein the report comprises a representation of said points corresponding to cardiac events located on said electrocardiogram and ballistocardiogram waveforms.

27. The apparatus as claimed in claim 24, wherein said parameters are selected from the group consisting of: time interval from one of said points corresponding to cardiac events, a valley on a ballistocardiogram waveform, a peak on a ballistocardiogram waveform, intersection of two ballistocardiogram waveforms, slope direction of a ballistocardiogram waveform and change of slope of a ballistocardiogram waveform.

28. The apparatus as claimed in claim 24, wherein said cardiac events are selected from the group consisting of: depolarization of the inter-ventricular septum (Q), atrial contraction (G), mitral valve close (H), isovolumic movement (I), rapid ejection period(J), aortic valve open (AVO), aortic valve close (AVC) and mitral valve open (M).

29. The apparatus as claimed in claim 10, further comprising an input device for receiving cardiac event identification input to produce an annotated heart beat, said input device in communication with said computer;
wherein said computer is configured for annotating electrocardiogram and ballistocardiogram waveforms by applying a template generated based on said cardiac event identification input, said electrocardiogram and ballistocardiogram waveforms correlated in time generated from said digital signals.

30. The apparatus as claimed in claim 29, wherein the report comprises a representation of annotated electrocardiogram and ballistocardiogram waveforms correlated in time.

31. The apparatus as claimed in claim 29, wherein said cardiac event identification input includes a reference event and other cardiac events.

32. The apparatus as claimed in claim 31, wherein said reference event is depolarization of the inter-ventricular septum (Q) or ventricular activation (R).

33. The apparatus as claimed in claim 31, wherein said other cardiac events are selected from the group consisting of: atrial contraction (G), mitral valve close (H), isovolumic movement (I), rapid ejection period (J), aortic valve open (AVO), aortic valve close (AVC) and mitral valve open (M).

34. A method for acquiring and analyzing data relating to a physiological condition of a subject, said method comprising:
detecting four analog signals using a sensor device coupled to said subject, one of said four analog signals being an electrocardiograph signal and three of said four analog signals being ballistocardiograph signals,
converting said four analog signals into digital signals;
transmitting data representative of said digital signals to a computer; and
performing an analysis of said data representative of said digital signals.

35. The method as claimed in claim 34, further comprising generating and outputting a report relating to said physiological condition.

36. The method as claimed in claim 34, wherein the ballistocardiograph signals are detected using a three-axis accelerometer comprised by said sensor.

37. The method as claimed in claim 34, further comprising providing commands to said sensor device to control detection of said four analog signals.

38. The method as claimed in claim 37, wherein said commands are provided by said computer, or by a portable terminal in communication with said sensor device and said computer.

39. The method as claimed in claim 34, further comprising associating subject identification information with said digital signals.

40. A sensor device for acquiring and transmitting signals relating to a physiological condition of a subject, said sensor device comprising:
a housing including a contact surface for coupling to a subject;
a three-axis accelerometer provided in said housing, said three-axis accelerometer for sensing vibrations of a chest wall of said subject;
conductive strips provided in said contact surface of said housing, said conductive strips being in communication with electrocardiograph lead circuitry for sensing cardiac electrical activity;
an analog to digital converter in communication with said three-axis accelerometer and said electrocardiograph lead circuitry, said analog to digital converter for receiving four analog signals, one of said four analog signals being an electrocardiograph signal and three of said four analog signals being ballistocardiograph signals corresponding to each axis of said three-axis accelerometer, and for converting said four analog signals into digital signals;
a power source; and
a transmitter provided in communication with said analog to digital converter for transmitting said digital signals.

41. The sensor device as claimed in claim 40, wherein one or more of said analog to digital converter, said power source and said transmitter are provided in said housing.

42. The sensor device as claimed in claim 40, wherein said transmitter is a radio device for wireless communication.

43. The sensor device as claimed in claim 40, wherein said transmitter transmits said digital signals to a computer or a portable terminal.

44. A method for locating and marking points on a waveform comprising:
providing data corresponding to electrocardiogram and ballistocardiogram waveforms correlated in time;
searching said data to locate points corresponding to cardiac events, a location of each of said points corresponding to cardiac events being defined by a rule set;
identifying said points corresponding to cardiac events; and
outputting a report comprising a representation of said points corresponding to cardiac events located on said electrocardiogram and ballistocardiogram waveforms.

45. The method as claimed in claim 44, wherein said rule set includes rules in which a location of each of said points corresponding to cardiac events is defined by at least one parameter.

46. The method as claimed in claim 45, wherein said at least one parameter is selected from the group consisting of: time interval from one of said points corresponding to cardiac events, a valley on a ballistocardiogram waveform, a peak on a ballistocardiogram waveform, intersection of two ballistocardiogram waveforms, slope direction of a ballistocardiogram waveform and change of slope of a ballistocardiogram waveform.

47. The method as claimed in claim 44, wherein said cardiac events are selected from the group consisting of: depolarization of the inter-ventricular septum (Q), atrial contraction (G), mitral valve close (H), isovolumic movement (I), rapid ejection period(J), aortic valve open (AVO), aortic valve close (AVC) and mitral valve open (M).

48. A computer-readable medium comprising instructions executable on a processor of a computer for implementing the method of claim 44.

49. A method for locating and marking points on a waveform comprising:
providing electrocardiogram and ballistocardiogram waveform data correlated in time and extending for at least two heart beats, one of said at least two heart beats being an annotated heart beat having cardiac events identified thereon, said cardiac events including a reference event located on an electrocardiogram waveform;
generating a template based on said annotated heart beat, said template including time intervals measured from said reference event to one or more other identified cardiac events; and
locating said reference event on one or more heart beats other than said annotated heart beat and applying said template to determine locations of said other cardiac events thereon.

50. The method as claimed in claim 49, further comprising optimizing a location of one or more of said other cardiac events.

51. The method as claimed in claim 50, wherein said location of said one or more of said other cardiac events is optimized by applying a rule.

52. The method as claimed in claim 51, wherein said rule moves a previously determined location of said one or more of said other cardiac events to an optimized location that coincides with a landmark located within a time window extending on either side of said previously determined location.

53. The method as claimed in claim 52, wherein said landmark is selected from the group consisting of: lowest point on a ballistocardiogram waveform, highest point on a ballistocardiogram waveform, intersection of two ballistocardiogram waveforms and smallest distance between two ballistocardiogram waveforms.

54. The method as claimed in claim 52, wherein said time window is about +/−10 ms.

55. The method as claimed in claim 49, wherein said reference event is depolarization of the inter-ventricular septum (Q) or ventricular activation (R).

56. The method as claimed in claim 49, wherein said other cardiac events are selected from the group consisting of: atrial contraction (G), mitral valve close (H), isovolumic movement (I), rapid ejection period (J), aortic valve open (AVO), aortic valve close (AVC) and mitral valve open (M).

57. A computer-readable medium comprising instructions executable on a processor of a computer for implementing the method of claim 49.
